(19) United States
(12) Patent Application Publication
(10) Pub. No.: US 2004/0148032 A1
(43) Pub. Date: Jul. 29, 2004

(54) Airway stent

(76) Inventors: Michael John Rutter, Cincinnati, OH (US); Robin Thomas Cotton, Cincinnati, OH (US); Jay Paul Willging, Cincinnati, OH (US)

Correspondence Address: HASSE GUTTAG & NESBITT LLC, 7550 CENTRAL PARK BLVD.,, MASON, OH, 45040, US

(21) Appl. No.: 10/353,318

(22) Filed: Jan. 29, 2003

(51) Int. Cl. A61F 2/04; A61F 2/20

(52) U.S. Cl. 623/23.7; 623/9

(57) ABSTRACT

An airways stent containing a hollow flexible tube and a plurality of domed pegs on the outer surface of at least the distal end of the tube to lift the tube off the airway passage wall. Also disclosed are intratracheal stents that have additional domed pegs on the outer surface of the proximal end of the tube, and suprastomal stents that have a removable plug in the proximal end of the tube to restrict airflow through the lumen of the tube.

### BACKGROUND OF THE INVENTION

The present invention relates to an airway stent useful for supporting an airway passage following surgical reconstruction, or to prevent obstruction of the airway. More particularly, the invention relates to an airway stent, such as an intratracheal stent or a suprastomal stent, comprising a hollow flexible tube having a plurality of domed pegs on the outer surface of at least the distal end of the tube.

Airway stents are designed to support the airway following surgical reconstruction or prevent obstruction of the airway due to tracheomalacia or tracheal compression. Although tracheotomy tubes and T-tubes may be considered airway stents, generally the term is used to describe either an intratracheal stent or a suprastomal stent. Intratracheal stents fall into two main categories, metal wire expandable stents and solid stents (which are usually hollow).

Metal wire stents have the advantage that they may be inserted into the airway while very thin, and then expanded to the desired diameter. They also do not significantly interrupt the normal mucociliary action of the trachea, and tend to maintain their position in the airway. However, the potential for granulation tissue formation and obstruction of the airway is a disadvantage. Moreover, removal of the stent may be difficult and involve an increased risk of complications.

Solid stents include the Aboulker/Cotton-Lorenz stent and the Dumon/Hood stents. The Aboulker/Cotton-Lorenz stent is typically used as a suprastomal stent following airway reconstruction. It lies above a tracheotomy tube, with the proximal end just above the vocal cords, and is not designed to be breathed through. The Aboulker/Cotton-Lorenz stent is typically held in place by a stitch passing through the stent. While an excellent stent, it is made of Teflon®, and availability is an increasing problem. If the stent is left in place for over six weeks, scarring may occur between the lower end of the stent and the tracheotomy tube.

The Dumon/Hood stent is a hollow silicone tube having cylindrical outer pegs intended to prevent migration of the stent in the trachea. The stent has a low propensity for granulation tissue formation. However, the pegs are not always sufficient to stabilize the stent, which is sometimes secured by a suture. Moreover, since it typically is a thick-walled stent designed primarily for adults, the stabilizing pegs can significantly reduce the size of the lumen available for respiration, especially in small stents required for children.

Thus, there is a need for an intratracheal stent that has a lumen large enough for comfortable respiration, even in small sizes used for children. The airway stent should also minimize granulation tissue formation and scarring, and should not interrupt the normal mucociliary action of the airway.

### BRIEF DESCRIPTION OF THE INVENTION

In one aspect, this invention provides an airway stent comprising:

(a) a hollow flexible tube having a proximal end, a distal end, and a continuous lumen between the proximal end and the distal end, said tube having a length and an outside diameter sized to provide an internal support for a reconstructed or corrected airway passage; and

(b) a plurality of domed pegs on the outer surface of the distal end of the tube to lift the distal end of the tube off the airway passage wall, said pegs having a generally circular base having a diameter of from about 1 to about 3 mm, a height of from about 0.5 to about 2 mm, and a ratio of height to diameter of less than about 0.7.

In another aspect, the invention provides an intratracheal stent comprising:

(a) a hollow flexible tube having a proximal end, a distal end, and a continuous lumen between the proximal end and the distal end, said tube having a length and an outside diameter sized to provide an internal support for a reconstructed or corrected trachea, and a wall thickness of less than about 2 mm; and

(b) a plurality of domed pegs on the outer surface of the proximal and distal ends of the tube to lift the proximal and distal ends of the tube off the tracheal wall, said pegs having a generally circular base having a diameter of from about 1 to about 3 mm, a height of from about 0.5 to about 2 mm, and a ratio of height to diameter of less than about 0.7.

Another aspect of the invention relates to a suprastomal stent comprising:

(a) a hollow flexible tube having a proximal end, a distal end, and a continuous lumen between the proximal end and the distal end, said tube having a length and an outside diameter sized to provide an internal support for a reconstructed or corrected larynx; and

(b) a plurality of domed pegs on the outer surface of the distal end of the tube to lift the distal end of the tube off the laryngeal wall, said pegs having a generally circular base having a diameter of from about 1 to about 3 mm, a height of from about 0.5 to about 2 mm, and a ratio of height to diameter of less than about 0.7.

### BRIEF DESCRIPTION OF THE DRAWINGS

The aspects and advantages of the invention will be better understood from the following detailed description, with reference to the accompanying drawings, in which:

FIG. 1 is a perspective view of an intratracheal airway stent of the invention;

FIG. 2 is a perspective view of an alternative intratracheal stent of the invention;

FIG. 3 is a perspective view of another intratracheal stent of the invention;

FIG. 4 is an expanded sectional view of the stent of FIG. 3, taken along line 4-4 in FIG. 3;

FIG. 5 is a partially cut-away perspective view of a suprastomal airway stent of the invention;

FIG. 6 is an expanded plan view of a plug suitable for insertion in the proximal end of the suprastomal stent shown in FIG. 5; and

FIG. 7 is an expanded plan view of the proximal end of the suprastomal stent shown in FIG. 5, with the plug shown in FIG. 6 inserted therein.

### DETAILED DESCRIPTION OF THE INVENTION

The airway stent of the present invention comprises a hollow flexible tube that has a proximal end, a distal end, and a continuous lumen therebetween. The stent also has a plurality of domed pegs on the outer surface of at least the distal end of the tube to lift the distal end off the airway passage wall. The domed pegs have a generally circular base having a diameter, height, and ratio of height to diameter selected to minimize granulation tissue formation and scarring from contact with the airway passage wall.

In one embodiment, the airway stent of the invention is an intratracheal stent useful for patients undergoing surgical reconstruction or repair of the trachea, or to support or prevent obstruction of the trachea. (As used herein, the term trachea includes the bronchus, and the term intratracheal includes tracheobronchial. A tracheobronchial stent herein may also have branching such as in the Y-shaped stents known in the art.) In another embodiment, the stent is a suprastomal stent for a patient undergoing reconstruction or repair of the larynx, or needing support for the larynx during healing. The airway stents of the present invention can be used for patients of all ages, and are especially useful for children undergoing airway surgery.

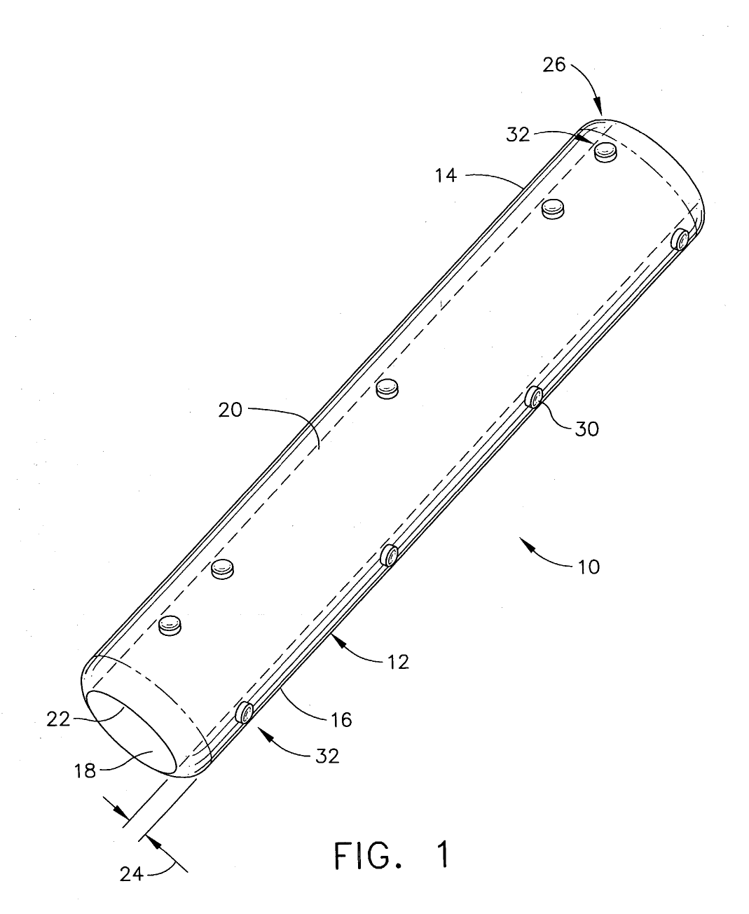

For a better understanding of the invention, reference is now made to FIG. 1 of the drawings. FIG. 1 illustrates an intratracheal stent 10 of the invention in the form of a hollow flexible tube 12 having a proximal end 14, a distal end 16, and a continuous lumen 18 extending between the proximal and distal ends. Tube 12 has an outer surface 20 and an inner surface 22 that together define the thickness 24 of the tube wall. The tube wall has a thickness of less than about 2 mm, typically from about 0.75 mm to about 1.5 mm, more typically about 1 mm. This relatively thin-walled stent allows the lumen to be large enough to provide adequate respiration, even with small intratracheal stents used for children.

In the embodiment shown in FIG. 1, both proximal end 14 and distal end 16 of tube 12 are inwardly curved as at 26 to reduce contact with and abrasion of the tracheal wall, and minimize tissue granulation and scarring.

The length and outside diameter of tube 12 are selected to provide internal support for a reconstructed or corrected trachea. The outside diameter thus varies as stent 10 is sized to meet requirements ranging from male adults to infants. By way of example, the outside diameter of tube 12 may be 6 or 8 mm for infants and up to 16 mm or 18 mm for adults. The length of tube 12 will also vary depending on whether stent 10 is intended for use on an adult or a child. However, the tube typically ranges in length from about 20 mm to about 50 mm for a child, and from about 20 mm to about 70 mm for an adult. Either end or both ends of tube 12 may be trimmed to obtain the desired length, as described hereinafter.

Stent 10 also contains a plurality of domed pegs 30 on the outer surface 20 of proximal end 14 and distal end 16 of tube 12. These lift the proximal and distal ends of the tube off the tracheal wall, thereby reducing granulation tissue formation and scarring from contact with the tracheal wall. The domed pegs have a generally circular base having a diameter of from about 1 to about 3 mm, typically from about 1.5 to about 2.5 mm, more typically about 2 mm. The domed pegs have a height (measured perpendicularly from outer surface 20 of tube 12) of from about 0.5 to about 2 mm, typically from about 0.75 to about 1.5 mm, more typically about 1 mm. The domed pegs also have a low profile, with a ratio of height to diameter less than about 0.7, typically less than about 0.6, more typically less than about 0.5. Such low profile, domed pegs provide minimal contact with the tracheal wall, thus reducing granulation tissue formation and scarring. In use, stent 10 is typically maintained in place by a transtracheal suture, although the domed pegs may also help to stabilize the stent.

In one embodiment, at least some of the domed pegs 30 are distributed around tube 12 in one or more bands 32 near the proximal and distal ends of the tube. Each such band contains at least three pegs per band, typically four pegs per band as in FIG. 1. (The third and fourth pegs in bands 32 are on the far side of tube 12 and hidden from view.) Such bands are typically located near the proximal and distal ends of tube 12 to lift the proximal and distal ends off the tracheal wall. In another embodiment, such as shown in FIG. 1, stent 10 contains additional domed pegs distributed on the outer surface 20 of tube 12 between its proximal end and distal end. These more centrally located domed pegs lift more centrally located sections of tube 12 off the tracheal wall, and help preserve the normal mucociliary action of the trachea. As also shown in FIG. 1, the domed pegs may be clustered near at least one, or both, of proximal end 14 and distal end 16 of tube 12 to allow the stent to be trimmed to the desired length, while still providing domed pegs at the resulting ends of the tube. In one embodiment, bands of domed pegs are clustered near the proximal and distal ends of the tube so that the stent may be trimmed to the desired length close to a band of pegs. After trimming, there is still at least one band of pegs at both the proximal and distal ends of the tube to lift the cut edges of the tube off the tracheal wall.

Stent 10 is preferably formed from a soft, resilient, biocompatible polymer material suitable for use in a living body, such as a silicone rubber that is relatively inert. The material typically has a surface energy close to that of the surrounding tissue, and dimensional stability sufficient to maintain its shape and to support the trachea. By utilizing a material having a surface energy similar to the surrounding tissue, rejection of the stent by the tissue is less likely to occur, thereby reducing the trauma experienced by the tissue. It will be appreciated that the composition of the material used will affect the resiliency, rigidity, and strength of the stent, and accordingly will affect the thickness of the wall necessary to support the trachea. In one embodiment, stent 10 is formed from resilient medical grade silicon marketed by the Dow Corning Corporation under the trade designations MDX44210 and C6-570. The outer surface 20 and inner surface 22 of tube 12 typically are smooth to deter adhesion of dust, mucus or moisture thereon and to minimize abrasion of the tissue mucosa. In general, frictional or chemical contact with the stent should be non-irritating to the surrounding tissue. A sintered coating is preferably applied to the surfaces of the stent to lessen the risk of granulation tissue formation.

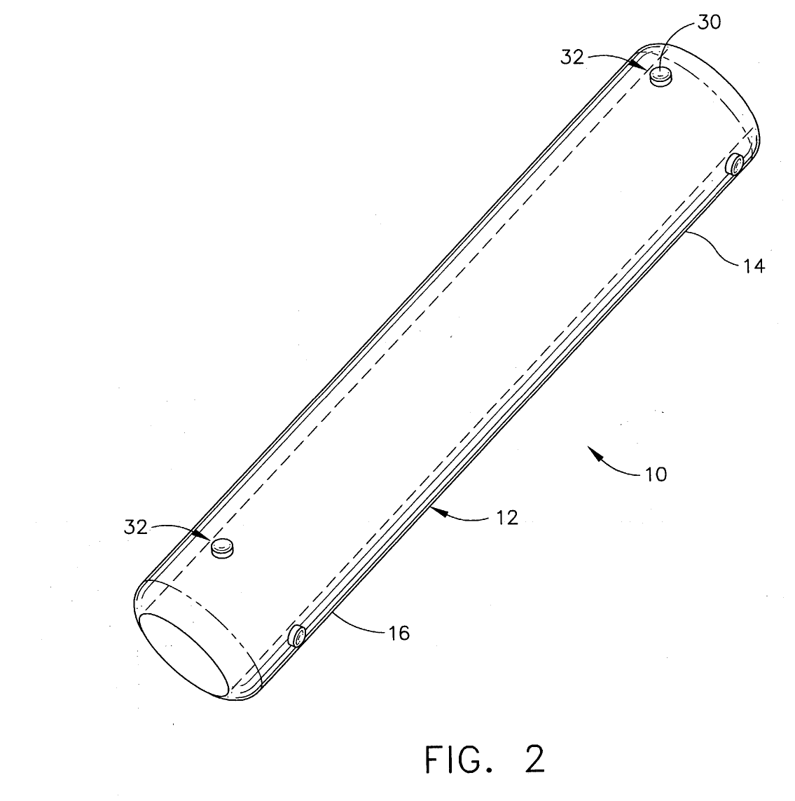

FIG. 2 shows an alternative intratracheal stent 10 of the invention containing bands 32 of domed pegs 30 only at proximal end 14 and distal end 16 of tube 12. This stent is otherwise similar to the stent shown in FIG. 1, and may be trimmed at either or both ends while still leaving the bands of domed pegs to lift the ends of the tube off the tracheal wall.

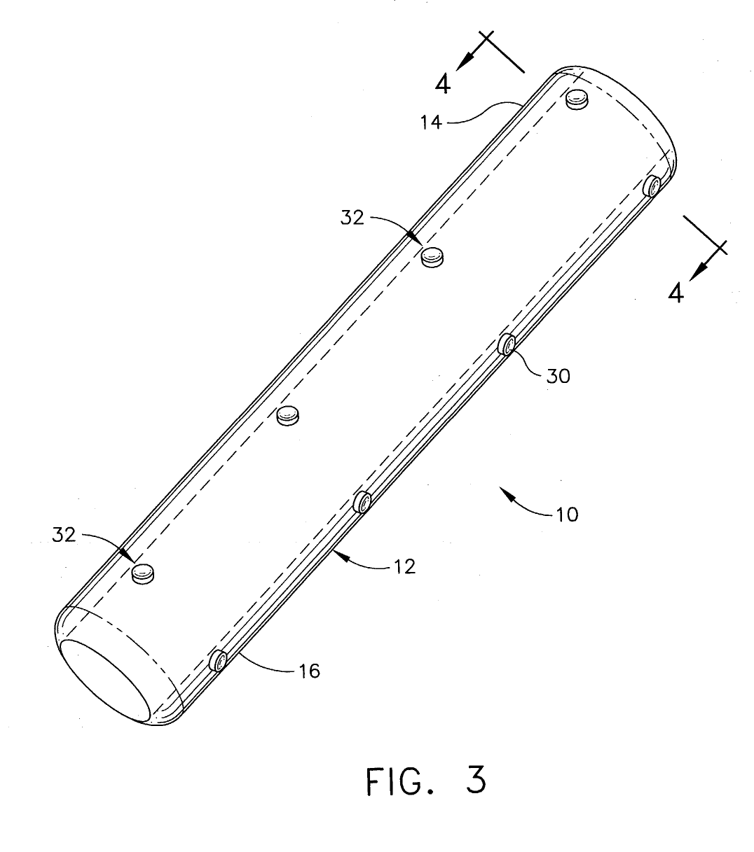

FIG. 3 is a perspective view of another intratracheal stent 10 of the invention containing bands 32 of domed pegs 30 distributed relatively uniformly along the length of tube 12. The bands at proximal end 14 and distal end 16 lift the proximal and distal ends of tube 12 off the tracheal wall. The more centrally located bands lift the more centrally located sections of tube 12 off the tracheal wall, helping to preserve the normal mucociliary action of the trachea.

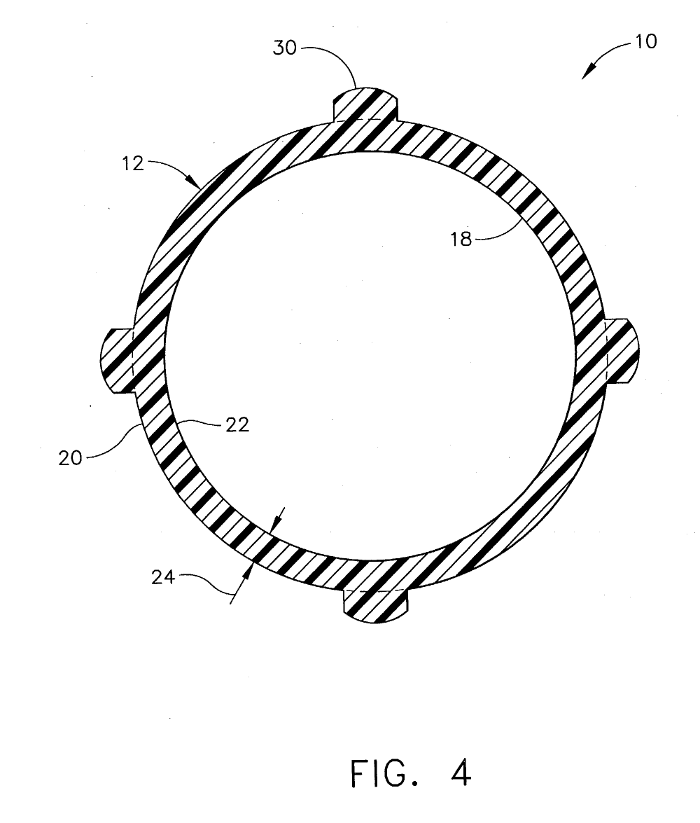

FIG. 4 is an expanded sectional view of stent 10 of FIG. 3, taken along line 4-4. In this embodiment, band 32 near proximal end 14 of tube 12 has four domed pegs 30 evenly distributed around outer surface 20 of tube 12. This sectional view shows the low profile nature of the domed pegs and the thin wall of tube 12, which together allow the lumen 18 of stent 10 to be relatively large when compared with that of other intratracheal stents containing pegs known in the art.

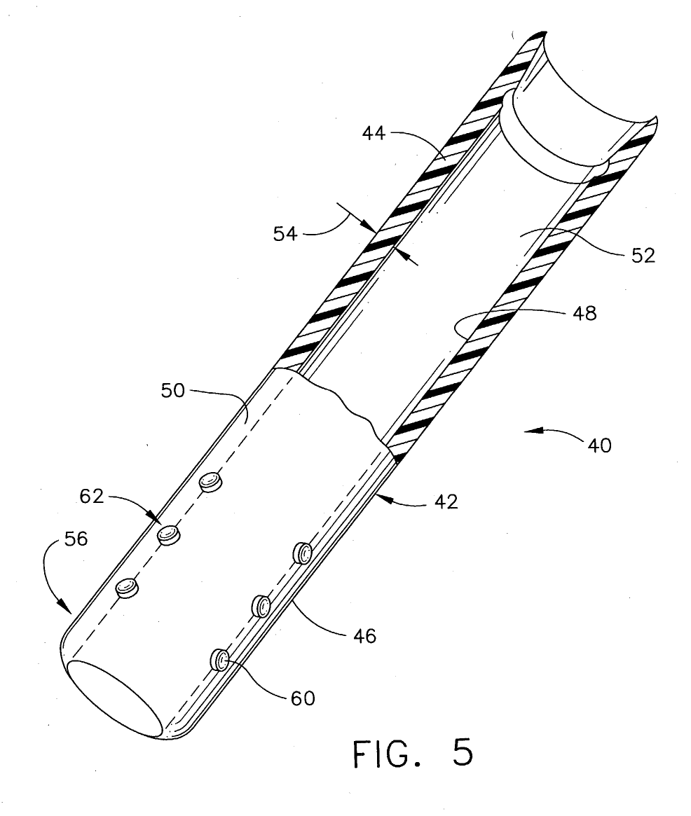

FIG. 5 is a partially cut-away perspective view of a suprastomal stent 40 of the present invention. Stent 40 is in the form of a hollow flexible tube 42 having a proximal end 44, a distal end 46, and a continuous lumen 48 extending between the proximal and distal ends. Tube 42 has an outer surface 50 and an inner surface 52 that together define the thickness 54 of the tube wall. The tube wall typically has a thickness of less than about 2 mm, more typically from about 0.75 mm to about 1.5 mm, usually about 1 mm. This relatively thin-walled stent is soft enough that once inserted, even if it overlaps the tracheotomy site, a tracheotomy tube can still be safely inserted to allow the patient to breath.

In the embodiment shown in FIG. 5, both proximal end 44 and distal end 46 of tube 42 are inwardly curved as at 56 to reduce contact with and abrasion of the laryngeal wall, and minimize tissue granulation and scarring.

The length and outside diameter of tube 42 are selected to provide internal support for a reconstructed or corrected larynx. The outside diameter thus varies as stent 40 is sized to meet requirements ranging from male adults to infants. By way of example, the outside diameter of tube 42 may be 6 or 8 mm for infants and up to 16 mm or 18 mm for adults. The length of tube 42 will also vary depending on whether stent 40 is intended for use on an adult or a child. However, the tube typically ranges in length from about 20 mm to about 50 mm for a child, and from about 20 to about 70 mm for an adult. The distal end 46 of tube 42 may be trimmed to obtain the desired length, as described hereinafter.

Stent 40 also contains a plurality of domed pegs 60 on the outer surface 50 of distal end 46 of tube 42. These lift the distal end of the tube off the laryngeal wall, thereby reducing granulation tissue formation and scarring from contact with the laryngeal wall. The domed pegs have a generally circular base having a diameter of from about 1 to about 3 mm, typically from about 1.5 to about 2.5 mm, more typically about 2 mm. The domed pegs have a height (measured perpendicularly from outer surface 50 of tube 42) of from about 0.5 to about 2 mm, typically from about 0.75 to about 1.5 mm, more typically about 1 mm. The domed pegs also have a low profile, with a ratio of height to diameter less than about 0.7, typically less than about 0.6, more typically less than about 0.5. Such low profile, domed pegs provide minimal contact with the laryngeal wall, thus reducing granulation tissue formation and scarring. In use, stent 40 is typically maintained in place by a transtracheal suture, although the domed pegs may also help to stabilize the stent.

In one embodiment, at least some of the domed pegs 60 are distributed around tube 42 in one or more bands 62 near the distal end 46 of the tube. Each such band contains at least three pegs per band, typically four pegs per band as in FIG. 5. (The third and fourth pegs in bands 62 are on the far side of tube 42 and hidden from view.) Such bands are typically located near the distal end 46 of tube 42 to lift the distal end off the laryngeal wall. In another embodiment, stent 40 contains additional domed pegs (which may or may not be in bands) distributed on the outer surface 50 of tube 42 between its proximal end 44 and distal end 46. These more centrally located domed pegs lift more centrally located sections of tube 42 off the laryngeal wall, and help preserve the normal mucociliary action of the larynx. However, domed pegs are not present at the proximal end 44 of tube 42 near the stomal opening. As shown in FIG. 5, the domed pegs may be clustered near the distal end 46 of tube 42 to allow the stent to be trimmed to the desired length, while still providing domed pegs at the distal end of the tube. In one embodiment, bands of domed pegs are clustered near the distal end of the tube so that the stent may be trimmed to the desired length close to a band of pegs. After trimming, there is still at least one band of pegs at the distal end of the tube to lift the cut edges of the tube off the laryngeal wall.

Stent 40 is preferably formed from a soft, resilient biocompatible polymer material suitable for use in a living body, such as a silicone rubber that is relatively inert. Stent 40 is typically made of a material that is soft enough that once it is inserted, even if it overlaps the tracheotomy site, a tracheotomy tube can still be safely inserted. The material typically has a surface energy close to that of the surrounding tissue, and dimensional stability sufficient to maintain its shape and to support the larynx. By utilizing a material having a surface energy similar to the surrounding tissue, rejection of the stent by the tissue is less likely to occur, thereby reducing the trauma experienced by the tissue. It will be appreciated that the composition of the material used will affect the resiliency, rigidity, and strength of the stent, and accordingly will affect the thickness of the wall necessary to support the larynx. In one embodiment, stent 40 is formed from resilient medical grade silicon marketed by the Dow Corning Corporation under the trade designations MDX44210 and C6-570. The outer surface 50 and inner surface 52 of the tube 42 typically are smooth to deter adhesion of dust, mucus or moisture thereon and to minimize abrasion of the tissue mucosa. In general, frictional or chemical contact with the stent should be non-irritating to the surrounding tissue. A sintered coating is preferably applied to the surfaces of the stent to lessen the risk of granulation tissue formation.

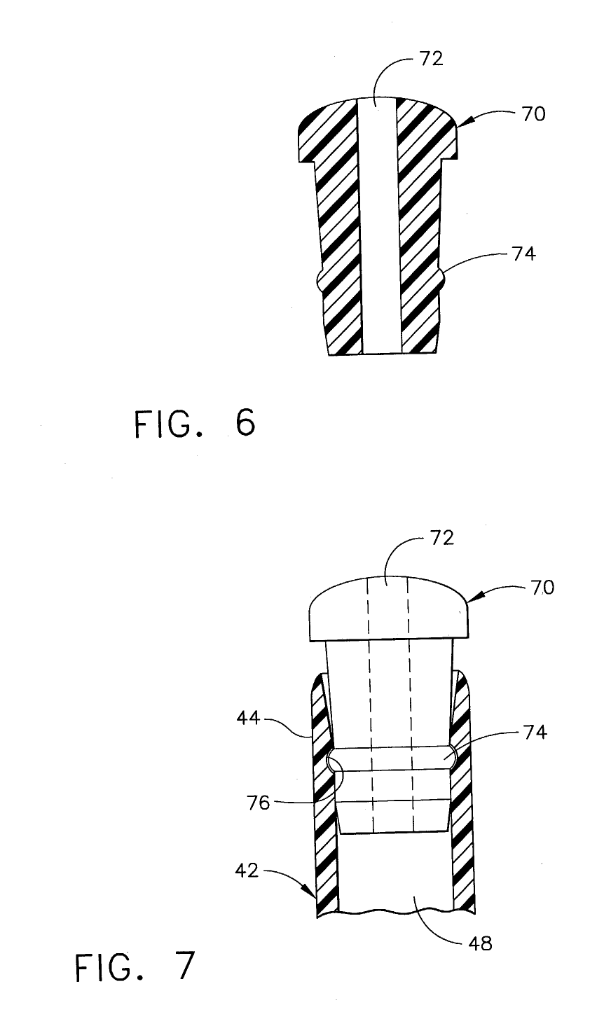

FIG. 6 shows an expanded plan view of a removable plug 70 that may be inserted in the proximal end 44 of tube 42 shown in FIG. 5 to restrict airflow through the continuous lumen 48 of the tube. Since suprastomal stent 40 is not designed to be breathed through, plug 70 minimizes aspiration through the lumen 48 of tube 42. Plug 70 may be a solid plug, or as shown in FIG. 6, it may have a narrow residual lumen 72 that provides a limited airway passage in the event of an emergency. Such a residual lumen typically has a diameter less than half, more typically less than a third, of the diameter of the lumen of tube 42. In one embodiment, as shown in FIG. 6, plug 70 has a flange 74 that grips into a molded or machined groove in the proximal end 44 of tube 42 to secure the plug in the proximal end of tube. Plug 70 is made of a biocompatible medical-grade silicone, and may be made of the same material as stent 40.

FIG. 7 is an expanded plan view of the proximal end 44 and continuous lumen 48 of tube 42 shown in FIG. 5, with plug 70 shown in FIG. 6 inserted therein. Proximal end 44 contains a molded grove 76 positioned to securely hold flange 74 of plug 70. As also shown, continuous lumen 48 has a diameter about three times that of residual lumen 72.

Although various embodiments of the invention have been described and exemplified, it will be understood that the scope of the invention is not limited to that description. Changes and modifications will occur to those of ordinary skill in the art and they can be made without departing from the spirit and scope of the invention. The invention is considered to include the methods of accomplishing the results described herein as well as structures designed to accomplish them.

As used herein, the term “comprising” means various components, capabilities and/or steps can be conjointly employed in the present invention. Accordingly, the term “comprising” encompasses the more restrictive terms “consisting essentially of” and “consisting of”.

What is claimed is:

1. An airway stent comprising:
(a) a hollow flexible tube having a proximal end, a distal end, and a continuous lumen between the proximal end and the distal end, said tube having a length and an outside diameter sized to provide an internal support for a reconstructed or corrected airway passage; and
(b) a plurality of domed pegs on the outer surface of the distal end of the tube to lift the distal end of the tube off the airway passage wall, said pegs having a generally circular base having a diameter of from about 1 to about 3 mm, a height of from about 0.5 to about 2 mm, and a ratio of height to diameter of less than about 0.7.

2. The airway stent of claim 1 wherein the pegs have a generally circular base having a diameter of from about 1.5 to about 2.5 mm and a height of from about 0.75 to about 1.5 mm.

3. The airway stent of claim 2 wherein the pegs have a ratio of height to diameter of less than about 0.6

4. The airway stent of claim 3 wherein the pegs have a ratio of height to diameter of less than about 0.5

5. The airway stent according to claim 1, further comprising a plurality of domed pegs on the outer surface of the proximal end of the tube to lift the proximal end of the tube off the airway passage wall.

6. The airway stent according to claim 5 wherein the tube has a wall thickness of less than about 2 mm.

7. The airway stent according to claim 6 wherein the pegs have a generally circular base having a diameter of from about 1.5 to about 2.5 mm and a height of from about 0.75 to about 1.5 mm.

8. The airway stent according to claim 7 wherein the pegs have a ratio of height to diameter of less than about 0.5

9. An intratracheal stent comprising:
(a) a hollow flexible tube having a proximal end, a distal end, and a continuous lumen between the proximal end and the distal end, said tube having a length and an outside diameter sized to provide an internal support for a reconstructed or corrected trachea, and a tube wall thickness of less than about 2 mm; and
(b) a plurality of domed pegs on the outer surface of the proximal and distal ends of the tube to lift the proximal and distal ends of the tube off the tracheal wall, said pegs having a generally circular base having a diameter of from about 1 to about 3 mm, a height of from about 0.5 to about 2 mm, and a ratio of height to diameter of less than about 0.7.

10. The intratracheal stent of claim 9 wherein the tube has a wall thickness of from about 0.75 mm to about 1.5 mm.

11. The intratracheal stent of claim 9 wherein the pegs have a generally circular base having a diameter of from about 1.5 to about 2.5 mm and a height of from about 0.75 to about 1.5 mm.

12. The intratracheal stent of claim 11 wherein the pegs have a ratio of height to diameter of less than about 0.6.

13. The intratracheal stent of claim 12 wherein the tube has a wall thickness of from about 0.75 mm to about 1.5 mm.

14. The intratracheal stent of claim 9 comprising pegs clustered in bands around the tube near the proximal and distal ends of the tube.

15. The intratracheal stent of claim 9 comprising additional domed pegs on the outer surface of the tube between its proximal and distal ends.

16. The intratracheal stent of claim 13 comprising pegs clustered in bands around the tube near the proximal and distal ends of the tube, and additional domed pegs on the outer surface to the tube between its proximal and distal ends.

17. A suprastomal stent comprising:
(a) a hollow flexible tube having a proximal end, a distal end, and a continuous lumen between the proximal end and the distal end, said tube having a length and an outside diameter sized to provide an internal support for a reconstructed or corrected larynx; and
(b) a plurality of domed pegs on the outer surface of the distal end of the tube to lift the distal end of the tube off the laryngeal wall, said pegs having a generally circular base having a diameter of from about 1 to about 3 mm, a height of from about 0.5 to about 2 mm, and a ratio of height to diameter of less than about 0.7.

18. The suprastomal stent of claim 17 wherein the pegs have a generally circular base having a diameter of from about 1.5 to about 2.5 mm and a height of from about 0.75 to about 1.5 mm.

19. The suprastomal stent of claim 18 wherein the pegs have a ratio of height to diameter of less than about 0.6.

20. The suprastomal stent of claim 19 comprising pegs clustered in bands around the tube near the distal end of the tube.

21. The suprastomal stent of claim 17 further comprising a removable plug in the proximal end of the tube that restricts airflow through the lumen of the tube.

22. The suprastomal stent of claim 21 wherein the plug has a flange that grips into a grove in the proximal end of the tube.

23. The suprastomal stent of claim 21 wherein the plug has a lumen having a diameter less than half the diameter of the lumen of the hollow flexible tube.

24. The suprastomal stent of claim 23 wherein the pegs have a generally circular base having a diameter of from about 1.5 to about 2.5 mm and a height of from about 0.75 to about 1.5 mm.

25. The suprastomal stent of claim 24 comprising pegs clustered in bands around the tube near the distal end of the tube.

26. The suprastomal stent of claim 25 wherein the plug has a flange that grips into a grove in the proximal end of the tube.
